(19) United States
(12) Patent Application Publication
(10) Pub. No.: US 2026/0014063 A1
(43) Pub. Date: Jan. 15, 2026

(54) Antiperspirant and Deodorant Composition

(71) Applicant: Jonathan Davies, Sheffield (GB)

(72) Inventor: Jonathan Davies, Sheffield (GB)

(21) Appl. No.: 18/998,139

(22) PCT Filed: Mar. 11, 2024
(86) PCT No.: PCT/IB2024/000107

§ 371 Date: Jan. 24, 2025

(30) Foreign Application Priority Data
Apr. 5, 2023 (GB) 2305054.5
Mar. 11, 2024 (WO) PCT/IB2024/000107

(51) Int. Cl. A61K 8/365; A61K 8/25; A61K 8/58; A61K 8/73; A61K 8/92; A61Q 5/12; A61Q 13/00; A61Q 15/00; A61Q 17/00; A61Q 19/00

(52) U.S. Cl. CPC A61K 8/365; A61K 8/25; A61K 8/585; A61K 8/732; A61K 8/922; A61Q 5/12; A61Q 13/00; A61Q 15/00; A61Q 17/005; A61Q 19/007

(57) ABSTRACT

The present invention relates to antiperspirant and deodorant compositions for correction of body odour, and more particularly relates to antiperspirant and deodorant compositions for human hygiene. The disclosed anti-perspirant and deodorant composition comprises isobutane, propane, butane, cyclopentasiloxane, caprylic/capric triglyceride, stearalkonium hectorite, propylene carbonate, c12-15 alkyl benzoate, parfum, silver lactate, lactic acid, aqua, zea mays starch, menthyl ethylamido oxalate, argania spinosa kernel oil. Furthermore, the active antimicrobial silver ingredient is dermatologically tested and is capable of diminishing odor-causing bacteria in addition to prevention of irritation and itching.

### CROSS-REFERENCE TO RELATED APPLICATIONS

No related applications are previously filed.

### TITLE OF THE INVENTION

Antiperspirant and Deodorant Composition

### TECHNICAL FIELD OF THE INVENTION

The present invention relates to antiperspirant and deodorant compositions for correction of body odour, and more particularly relates to antiperspirant and deodorant compositions for human hygiene.

### BACKGROUND OF THE INVENTION

Deodorants and antiperspirants are personal care products made to reduce or get rid of perspiration and body odour. Antiperspirants operate by blocking sweat glands to limit perspiration production, whereas deodorants hide or neutralise the odour caused by bacteria that live on sweat.

Typically, deodorants and antiperspirants combine active chemicals with other elements to provide the desired benefits. Antimicrobial compounds like triclosan or phenoxyethanol, which destroy the germs that produce odour, are commonly found as the active components in deodorants. For added benefits, other substances like moisturisers and fragrances may also be used.

On the other hand, antiperspirants contain active chemicals such as aluminium salts that limit perspiration production by producing a blockage in the sweat ducts. For added benefits, other substances including skin conditioners and fragrances may also be used. In order to appeal to consumers, deodorants and antiperspirants are frequently created with aromas that are viewed reflecting different aroma. To further underline their efficacy, they may also be advertised with different phrases.

Some customers prefer deodorants that are natural or free of aluminium and that use chemicals like baking soda, cornstarch, or essential oils to lessen odour and perspiration. These products might be promoted as healthier or more environmentally friendly substitutes for more conventional deodorants and antiperspirants.

Human intimate hygiene specific antiperspirant and deodorant products are growing in popularity as more people look for solutions to problems like odour, sweating, and chafing in sensitive areas. Antiperspirants for intimate areas frequently have active ingredients such as aluminium chloride, which limit perspiration formation by blocking sweat glands. Deodorants made for private regions frequently include antimicrobial ingredients like triclosan or chlorhexidine, which eliminate odor-causing microorganisms. Aloe vera and witch hazel are examples of additional ingredients that may be used to give the skin additional advantages like relaxing and moisturising effect.

There are several drawbacks related to using antiperspirant and deodorant compositions in intimate areas for humans. The skin in the intimate area is sensitive and can be easily irritated by certain ingredients found in antiperspirants and deodorants, such as alcohol, fragrances, and certain antimicrobial agents. This can cause redness, itching, and discomfort.

Antimicrobial agents found in deodorants and antiperspirants can disrupt the natural bacterial flora in the intimate area, potentially leading to an overgrowth of harmful bacteria and increased risk of infection. There is some concern that certain ingredients in antiperspirants and deodorants, such as aluminum salts, may be absorbed by the body and contribute to health problems such as Alzheimer's disease. However, more research is needed to confirm these claims. Antiperspirants and deodorants may not be as effective in the intimate area, as the skin is often damp and subject to friction, which can reduce the product's ability to adhere to the skin and provide protection.

There are very few products in the market that are specifically formulated for use in intimate areas and contain gentle, pH-balanced ingredients that won't irritate the skin. Fragrance-free products may be especially helpful in avoiding irritation, however, such anti-perspirant and deodorant compositions still have their drawbacks. It is key to note that whilst existing antiperspirant deodorants can be sprayed onto the intimate area by choice of the user, this type of usage comes with the imposed risks that are mentioned above, thus they cannot be recommended or marketed for intimate usage as they have not been specially formulated and tested as such, whereas the disclosed invention has been formulated and tested to meet the satisfactory requirements of both of these criteria.

In view of this, various solutions have been proposed, none combine the characteristics of the present invention. Thus, there is a need for an anti-perspirant and deodorant composition that is safe to use at intimate areas and can diminish the odour causing bacteria.

### SUMMARY OF THE INVENTION

Embodiments of the invention solve the above-mentioned problems by providing an anti-perspirant and deodorant composition. The disclosed anti-perspirant and deodorant composition comprises isobutane, propane, butane, cyclopentasiloxane, caprylic/capric triglyceride, stearalkonium hectorite, propylene carbonate, c12-15 alkyl benzoate, parfum, silver lactate, lactic acid, aqua, zea mays starch, menthyl ethylamido oxalate, argania spinosa kernel oil. Furthermore, the active antimicrobial silver ingredient is dermatologically tested and is capable of diminishing odor-causing bacteria in addition to prevention of irritation & itching.

This summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other aspects, and advantages of the subject matter described herein will become apparent from the following detailed description, and claims.

### BRIEF DESCRIPTION OF THE DRAWINGS

Drawings are not required as the subject matter of the present invention is related to a composition.

### DETAILED DESCRIPTION OF THE INVENTION

The following detailed description are intended to describe aspects of the invention in sufficient detail to enable those skilled in the art to practice the invention. Other embodiments can be utilized, and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, embodiments of the invention can include a variety of combinations and/or integrations of the embodiments described herein.

The present invention discloses an anti-perspirant and deodorant composition comprises isobutane, propane, butane, cyclopentasiloxane, caprylic/capric triglyceride, stearalkonium hectorite, propylene carbonate, c12-15 alkyl benzoate, parfum, silver lactate, lactic acid, aqua, zea mays starch, menthyl ethylamido oxalate, argania spinosa kernel oil. Furthermore, the active antimicrobial silver ingredient is dermatologically tested and is capable of diminishing odor-causing bacteria in addition to preventing irritation and itching.

In one embodiment, the present invention comprises propellant made from well selected mixtures of isobutane, butane, and propane that has been readily purified and deodorised using various hydrogenation and separation procedures. Its carefully balanced composition ensures the appropriate gas pressure for each application. These products are especially designed to function as expansion gases in polymeric materials and as propellants in aerosols for packaged products for a variety of applications.

In the same embodiment, the composition further comprises Cyclopentasiloxane as a hair or skin conditioning agent. Cyclopentasiloxane is a silicone that is commonly found in cosmetics. It is frequently present in lubricants, sealants, medical implants, and windscreen coatings. It is water-thin, odourless, colorless, and non-greasy. It does not penetrate the skin. Instead, it quickly evaporates away from it. Its property to dry fast makes it a valuable ingredient for cosmetics like antiperspirants and hair sprays.

In the same embodiment, the composition further comprises Bentone Gel® GTCC V that further includes Capric Triglyceride, Stearalkonium Hectorite, and Propylene Carbonate. Specifically, Capric Triglyceride acts as a fragrance, perfuming and skin condition agent whereas Stearalkonium Hectorite acts as a gel forming and viscosity controlling agent and Propylene Carbonate acts as solvent viscosity controlling agent.

In the same embodiment, the composition further comprises C12-15 alkyl benzoate that functions as an emollient (skin-softening, moisture-sealing), texture-enhancing and dispersing agent. In addition, C12-15 alkyl benzoate also has antimicrobial properties.

In the same embodiment, the composition further comprises parfum that has deodorant, masking, and perfuming function.

In the same embodiment, the composition further comprises silver lactate, lactic acid and aqua in which silver lactate acts as a deodorant, lactic acid has buffering humectant and skin conditioning function and aqua is used as a solvent.

In the same embodiment, the composition further comprises Zea Mays Starch and Aqua in which Zea Mays Starch has abrasive absorbent anticaking and viscosity controlling properties whereas aqua is use as solvent. One of the major functions of Zea Mays Starch is to make deodorant feel dry (rather than wet or slippery) as its applied.

In the same embodiment, the composition further comprises Menthyl Ethylamido Oxalate for skin conditioning.

In the same embodiment, the composition further comprises Argania Spinosa Kernel Oil that has emollient and skin conditioning properties.

In a preferred embodiment, an anti-perspirant and deodorant composition comprising: propellants that further includes 65.4356% w/w w/w of isobutane, 16.4000% w/w of propane, and 0.1640% w/w of butane; 8.2000% w/w of cyclopentasiloxane that has hair conditioning, skin conditioning, emollinent, and solvent properties; 3.4960% w/w of caprylic/capric triglyceride that has fragrance, skin conditioning, perfuming properties; 0.2920% w/w of stearalkonium hectorite that has gel forming and viscosity controlling properties; 0.2120% w/w of propylene carbonate that has solvent and viscosity controlling properties; 2.0000% w/w of c12-15 alkyl benzoate that has antimicrobial, emollient and skin conditioning properties; 0.2000% w/w of parfum that has deodorant, masking and perfuming properties; 0.0023% w/w of silver lactate that has deodorant properties; 0.0023% w/w of lactic acid that has buffering, humectant, and skin conditioning properties; 0.4955% w/w of aqua that act as solvent; 1.7400% w/w of zea mays starch that has abrasive, absorbent, anticaking, skin protecting, and viscosity controlling properties, and 0.2600 of aqua that acts as solvent; 0.1000% w/w of menthyl ethylamido oxalate that has skin conditioning properties; and 1.0000% w/w of argania spinosa kernel oil that further has emollient and skin conditioning properties.

Although the description above contains many specifics, these should not be construed as limiting the scope of the embodiments but as merely providing illustrations of some of several embodiments.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular examples of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Having thus described in various embodiments of the invention, what is claimed as new and desired to be protected by Letters Patent includes the following:

1. An anti-perspirant and deodorant composition comprising:
a. propellant mixture that further includes 65.4356% w/w of isobutane, 16.4000% w/w of propane, 0.1640% w/w of butane,
b. 8.2000% w/w of cyclopentasiloxane that has hair conditioning, skin conditioning, emollinent, and solvent properties;
c. 3.4960% w/w of caprylic/capric triglyceride that has fragrance, skin conditioning, perfuming properties;
d. 0.2920% w/w of stearalkonium hectorite that has gel forming and viscosity controlling properties; 0.2120% w/w of propylene carbonate that has solvent and viscosity controlling properties;
e. 2.0000% w/w of c12-15 alkyl benzoate that has antimicrobial, emollient and skin conditioning properties;
f. 0.2000% w/w of parfum that has deodorant, masking and perfuming properties;
g. 0.0023% w/w of silver lactate that has deodorant properties;
h. 0.0023% w/w of lactic acid that has buffering, humectant, and skin conditioning properties;
i. 0.4955% w/w of aqua that act as solvent;
j. 1.7400% w/w of zea mays starch that has abrasive, absorbent, anticaking, skin protecting, and viscosity controlling properties, and 0.2600 of aqua that acts as solvent;
k. 0.1000% w/w of menthyl ethylamido oxalate that has skin conditioning properties; and
l. 1.0000% w/w of argania spinosa kernel oil that further has emollient and skin conditioning properties.

2. The anti-perspirant and deodorant composition of claim 1, wherein the composition is used on intimate area of human body.

3. The anti-perspirant and deodorant composition of claim 1, wherein the propellant mixture is purified and deodorized using hydrogenation and separation procedure.
